(19) United States
(12) Patent Application Publication
(10) Pub. No.: US 2021/0205053 A1
(43) Pub. Date: Jul. 8, 2021

(54) IMPLANT DENTISTRY METHODS AND APPARATUSES

(71) Applicant: Joel L. Rosenlicht, Naples, FL (US)

(72) Inventor: Joel L. Rosenlicht, Naples, FL (US)

(21) Appl. No.: 17/142,714

(22) Filed: Jan. 6, 2021

Related U.S. Application Data
(60) Provisional application No. 62/957,956, filed on Jan. 7, 2020.

(51) Int. Cl. A61C 8/00; A61B 17/16

(52) U.S. Cl. CPC A61C 8/0089; A61B 17/1673; A61B 17/1615

(57) ABSTRACT

A drill kit includes at least one shaft of a first connection type, each shaft having a different length than each other shaft of the at least one shaft of the first connection type. The drill kit further includes preparation drill bits each having a first connection geometry. The drill kit further includes compression drill bits each having the first connection geometry. Each shaft has a proximal end configured to connect with an actuator instrument and a distal end configured to connect to the first connection geometry of each of the preparation drill bits and to the first connection geometry of each of the compression drill bits. The devices and methods may optimize the ability to choose surgical access and limit needing to handle components for a touchless and more sterile or hygienic procedure and protocol.

### CROSS-REFERENCE TO RELATED APPLICATION

This application claims the benefit of U.S. Provisional Patent Application No. 62/957,956, filed on Jan. 7, 2020, which is hereby incorporated by reference in its entirety.

### TECHNICAL FIELD

The present disclosure generally relates to drilling and, more particularly, drilling for implant dentistry and medical/surgical bone drilling applications.

### BACKGROUND

In implant dentistry, conventional drills and methods are used to remove bone material of a patient's jaw in order to optimize the site for stability for a dental implant. The spectrum of dental drills (burrs) in design, materials and application is extensive. Over the years, many new developments in dental drills (burrs) have occurred in the field of dentistry for the various disciplines. The designs have occurred to fit specific needs, e.g. dental implants, drilling for site preparation, harvesting of bone, or for routine dental restorative or surgical treatments.

Dental implant drills on the market today are, for the most part, designed for the purpose of site preparation or surgery, without a major consideration of the environment in which the drill is being used.

In dentistry, the actuator instrument or hand-piece of the drill and drill bit are positioned within the patient's mouth. This environment presents unique and common challenges. The inability of a patient to open their mouth sufficiently wide to accommodate the hand-piece, drill and dentist's hand make drilling with standard drills difficult in addition to the challenge of accommodating anatomical variations of bone in relation to teeth between patients. Conventional drills are manufactured at different lengths to accommodate different dental implants, but the significant number of drills and/or drill extensions required to accomplish the task of selecting the appropriate drill size is still often inadequate. Conventional drill shafts require drill shaft extenders in order to change the length of a shaft, which make the drill more wobbly and unstable. It is known that conventional drill bits in the dentistry field have integral shafts that connect to a drill directly (or other actuator) or connects to the drill through a shaft extender.

### SUMMARY

The present disclosure provides a drill system where the drill (burr) has a relatively small connection interface (e.g. a male interface). The drill system (or kit) may be configured to prepare osteotomies for numerous dental implant systems. The drill system includes a variety of shafts of different lengths that are configured to quick change with an actuator instrument (e.g. a motor-equipped instrument) in order to suit the anatomical need of the patient's surgical site. Each shaft may be configured to connect to an attachment shaft of the actuator instrument or to an interface of the actuator instrument.

Drills according to the present disclosure may be configured to remove dense cortical bone, but allow for a thinner dimension into the medullary bone so that when the dental implant is to be placed, there may be greater compaction, densification and initial stability.

The drills and shafts may be arranged in a universal implant drilling system (or kit). The universal drilling system could accommodate many different kinds of implant systems. The universal implant drilling system may advantageously have fewer components than conventional drill systems and be configured for: implant osteotomies; site preparation for implant placement; harvesting of autogenous bone; provide for bone densification in conjunction with implant site preparation. Drill components may also be configured to be used with conventional actuator instruments. The universal implant drilling system may allow for easy access to individual drills and their usage for recording how many times the burrs have been used for auto replacement.

The system may be rigid, secure, and easy to separate in the kit.

### BRIEF DESCRIPTION OF THE DRAWINGS

FIG. 1 is a diagram of a preparation drill bit in accordance with the present disclosure;

FIG. 2 is a diagram of a universal preparation drill system in accordance with the present disclosure;

FIG. 3 is a diagram of a compression drill bit in accordance with the present disclosure;

FIG. 4 is a diagram of a universal compression drill system in accordance with the present disclosure;

FIG. 5 is a diagram of a universal preparation and compression drill system in accordance with the present disclosure;

FIG. 6 is a flow diagram of a method in accordance with the present disclosure;

FIG. 7 is a diagram of a drill method in accordance with the present application;

FIG. 8 is a diagram of a drill bit with connection geometry in accordance with the present disclosure;

FIG. 9 is a diagram of a drill bit with connection geometry in accordance with the present disclosure;

FIG. 10A is a diagram of a plurality of actuator instrument shafts in accordance with the present disclosure;

FIG. 10B is a diagram of a plurality of actuator instrument shafts in accordance with the present disclosure;

FIG. 11 is a diagram of a bone harvesting drill bit in accordance with the present disclosure;

FIG. 12 is a diagram of drill bits with different diameters in accordance with the present disclosure;

FIG. 13 is a diagram of a drill kit in accordance with the present disclosure;

FIG. 14 is a diagram of another drill kit in accordance with the present disclosure; and

FIG. 15 is a diagram of a dental implant in accordance with the present disclosure.

### DETAILED DESCRIPTION

Before the various embodiments are described in further detail, it is to be understood that the present disclosure is not limited to the particular embodiments described. It will also be understood that the methods and apparatuses described herein may be adapted and modified as appropriate for the application being addressed and that the devices, systems, kits and methods described herein may be employed in other suitable applications, and that such other additions and modifications will not depart from the scope thereof.

Although various features have been shown in different figures for simplicity, it should be readily apparent to one of skill in the art that the various features may be combined without departing from the scope of the present disclosure.

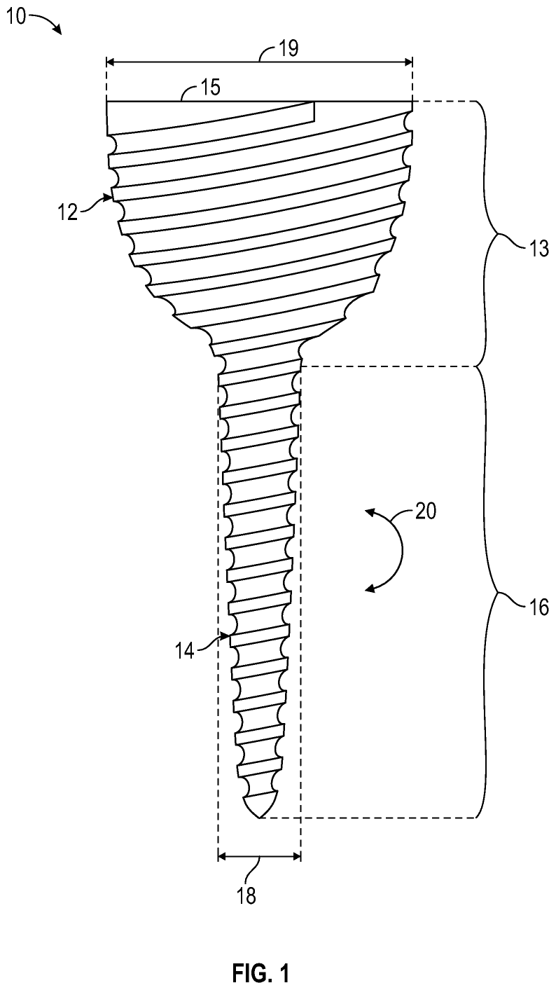

Referring to FIG. 1, an exemplary preparation drill bit (or burr) 10 in accordance with the present disclosure is shown. The preparation drill bit 10 includes a base portion 12 connected to a tip portion 14 having a substantially conical form. The base portion has a length 13 and a diameter (or width) 15. The tip portion 14 has a length 16. The tip portion 14 has a smaller width 18 than the diameter (or width) 15 of the base portion 12 in a width direction 19. The base portion 12 and/or the tip portion 14 are configured to remove bone when rotated in a rotation direction 20. While the tip portion 14 has a generally conical shape or form, other shapes or forms configured to remove bone (or other material) are within the scope of the present disclosure.

The preparation drill bit 10 may be used as an initial preparation drill to begin the removal of bone at a surgical site in the jaw of a patient. When drilling into the bone of a patient with the preparation drill bit 10, an initial bone cavity can be formed at a depth equal to the sum of the length 13 of the base portion 12 and the length 16 of the tip portion 14, or the length 16 of the tip portion 14 only, or some intermediate depth in between.

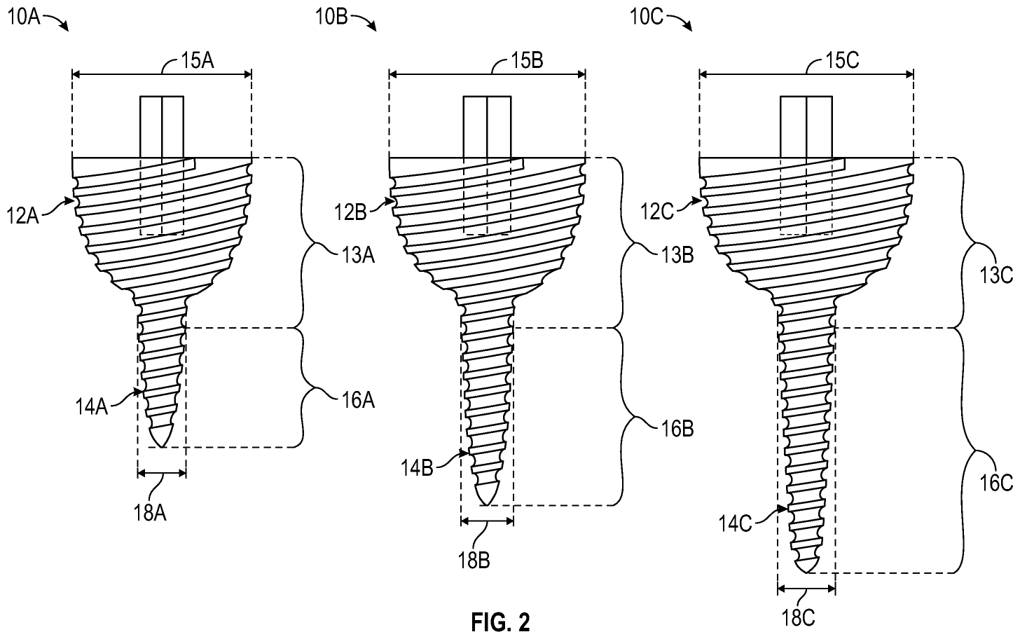

Referring to FIG. 2, a universal preparation drill system (or kit) 24 in accordance with the present disclosure includes a plurality of preparation drill bits 10A, 10B, 10C (collectively or generally 10). Each preparation drill bit 10 has a base portion 12A, 12B, 12C (collectively or generally 12). Each preparation drill bit 10 has a different tip portion 14A, 14B, 14C (collectively or generally 14) having different lengths 16A, 16B, 16C, respectively. Each preparation drill bit 10 has a different diameter (or width) 15A, 15B, 15C. While three preparation drill bits 10 are shown, it is within the scope of the present disclosure to have any number of preparation drill bits 10 and/or any number or length of tip portions 14. In FIG. 2, the base portions 12 of the preparation drill bits 10 are the same size. However, in some embodiments, the base portions 12 are different sizes.

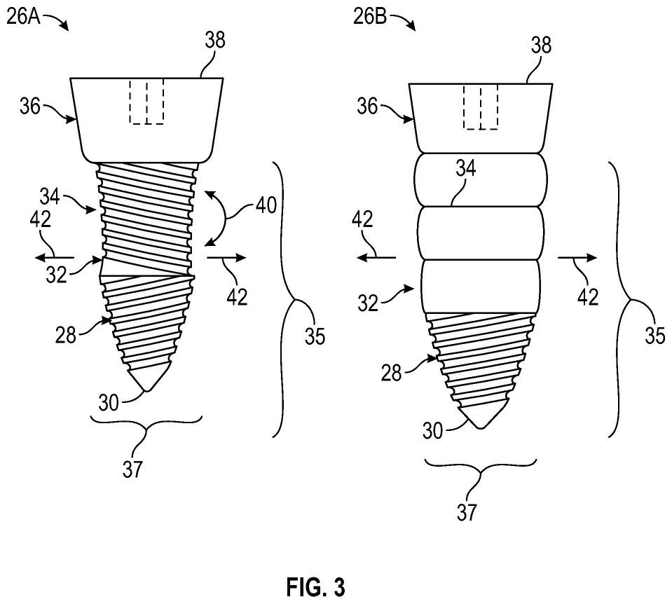

Referring to FIG. 3, compression drill bits (or burr) 26A, 26B (collectively or generally 26) in accordance with the present disclosure are shown. The compression drill bits 26 include a cutting tip 28 at a distal end 30, a compression section 32 having compression geometry 34 on the exterior surface thereof, and a base portion 36 at a proximal end 38. The cutting tip 28 is configured to cut and remove bone when the compression drill bit 26A, 26B is rotated in a rotation direction 40. The compression geometry 34 is configured force bone fragments that have been cut or removed by the cutting tip 28 in a radial direction 42 when the compression drill bit 26A, 26B is rotated in the rotation direction 40. In practice, bone fragments may not move only in the radial direction 42, but will have a vector component of movement in the radial direction 42 with enough force to compact the bone fragment into a sidewall of the bone cavity, as is discussed in greater detail herein. In some embodiments, in addition to compressing by forcing bone fragments in a radial direction (or in alternative thereof), bone compression at the compression section is accomplished by contact with the compression section with the side wall of the bone in an initial bone cavity that is prepared by a preparation drill bit(s). For example, the compression geometry 34 of compression drill bit 26B can force bone at the bone side wall of an inner cavity in a radial outward direction to compress, with or without also compressing bone fragments into the bone side wall. In some embodiments, the groove/flute pattern at the cutting tip 28 is different than the groove/flute pattern at the compression section 32, e.g. the groove/flute pattern changes direction as shown in FIG. 3. The compression drill bit 26A has a concave compression geometry 34 while the compression drill bit 26B has a variable increasing compression geometry 34 (increasing in diameter in a direction towards the base portion 36) with thread grooves.

The cutting tip 28 and compression section 32 of the compression drill bit 26 have a combined length 35 and a maximum width 37 at the widest point. The ratio of combined length 35 and maximum width 37 may be virtually any ratio depending on the desired cutting and compression characteristics of the compression drill bit 26 and/or the anatomical factors of the patient, as is discussed in greater detail herein.

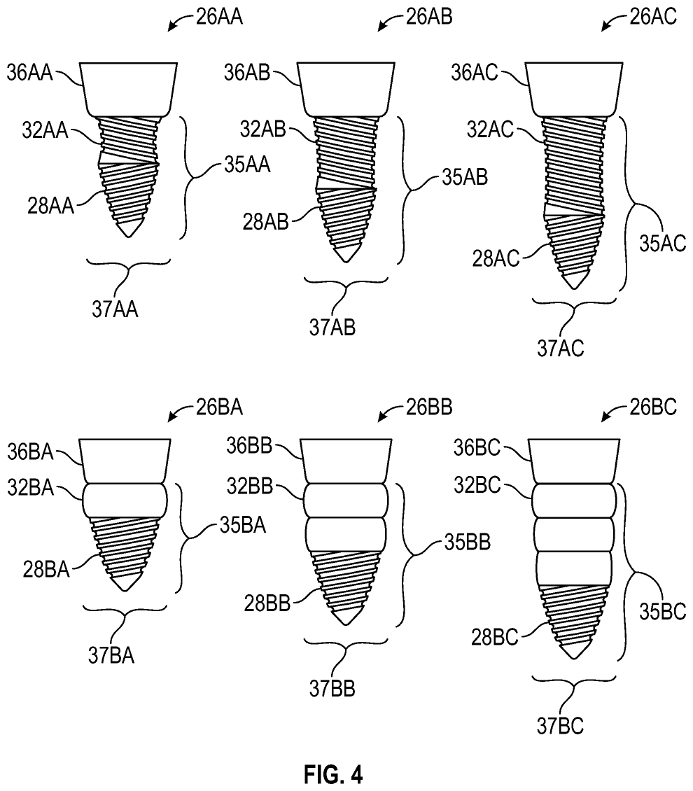

Referring to FIG. 4, a universal compression drill system 44 in accordance with the present disclosure includes a plurality of compression drill bits 26AA, 26AB, 26AC, 26BA, 26BB, 26BC (collectively or generally 26). Each compression drill bit 26 has a different cutting tip 28AA, 28AB, 28AC, 28BA, 28BB, 28BC and compression section 32AA, 32AB, 32AC, 32BA, 32BB, 32BC having different combined lengths 35AA, 35AB, 35AC, 35BA, 35BB, 35BC (collectively or generally 35), respectively. While six compression drill bits 26 are shown, it is within the scope of the present disclosure to have any number of compression drill bits 26 and/or any number or combined lengths 35 of cutting tips 28 and compression sections 32. In FIG. 4, the base portions 36AB, 36AB, 36AC, 36BA, 36BB, 36BC (collectively or generally 36) of the compression drill bits 26 are the same size. However, in some embodiments the base portions 36 are different sizes. While the universal compression drill system 44 is shown with compression drill bits 26 having concave compression geometry 32 (i.e. compression drill bits 26AA, 26AB, 26AC) and compression drill bits 26 having straight compression geometry 32 (in contrast to the variable increasing compression geometry of 26B in FIG. 3) (i.e. compression drill bits 26BA, 26BB, 26CC), in some embodiments a universal compression drill system may comprise only drill bits 26 having concave compression geometry 32 or only compression drill bits 26 having straight compression geometry 32. In some embodiments, one or more compressions drill bits 26 may have convex compression geometry 32.

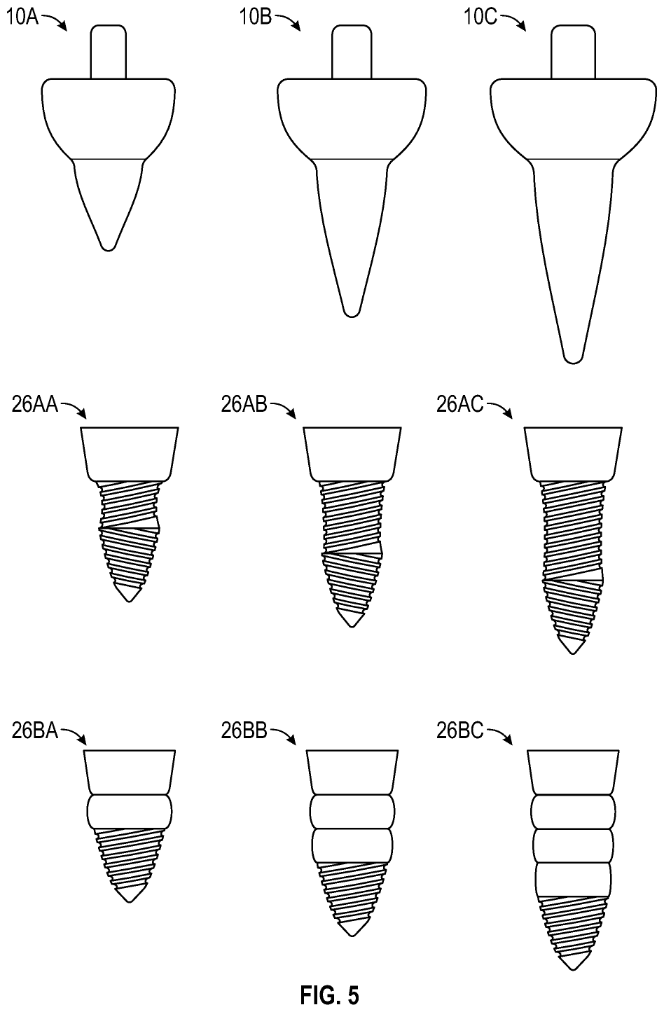

Referring to FIG. 5 a universal preparation and compression drill system 46 in accordance with the present disclosure is shown. The universal preparation and compression drill system 46 includes the universal preparation drill system 24 of FIG. 2 and the universal compression drill system 44 of FIG. 4. While the universal preparation and compression drill system 46 is shown as having a different number of preparation drill bits 10 as compression drill bits 26, in some embodiments the number of preparation drill bits 10 is the same number of compression drill bits 26. In some embodiments, a compression drill bit 26 may correspond to one or more preparation drill bits 10 based on width and length of the compression drill bit 26 and preparation drill bit 10. For example, in some embodiments, a compression drill bit 26 may correspond to a preparation drill bit 10 if the compression drill bit 26 has a cutting tip 28 and compression section 32 combined length 35 being the same as the length 16 of the tip portion 14 of the preparation drill bit 10 or the sum of the length 13 of the base portion 12 and the length 16 of the tip portion 14, or within a range, such as and without limitation, within 2 millimeters, 4 millimeters, 6 millimeters, 8 millimeters or 10 millimeters. In some embodiments, a compression drill bit 26 may correspond to a preparation drill bit 10 if the compression drill bit 26 has a maximum width 37 being the same as the width 18 of the tip portion 14 of the preparation drill bit 10, or within a range, such as and without limitation, within 2 millimeters, 4 millimeters, 6 millimeters, 8 millimeters or 10 millimeters. The range(s) may be configured based on considerations of the application being addressed, such as, for example, bone quality, localization, etc.

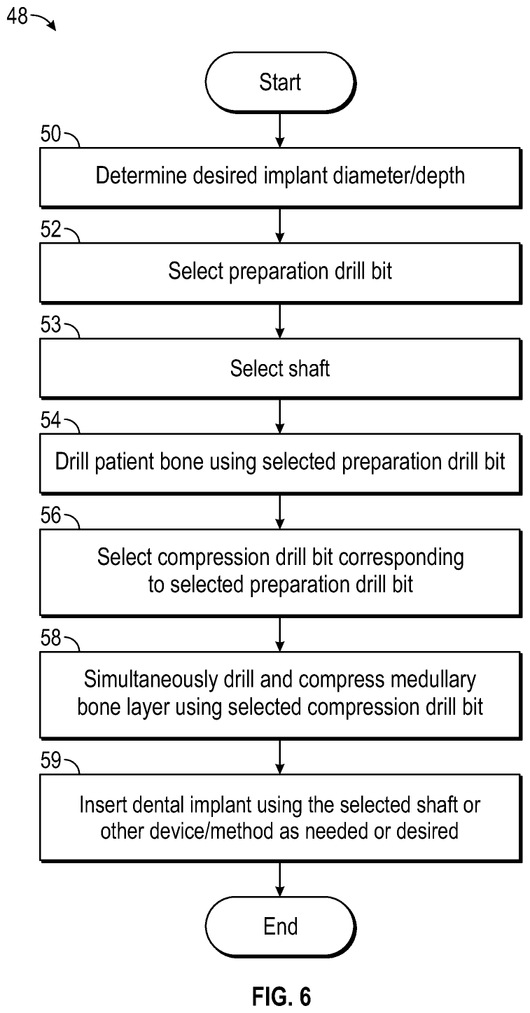

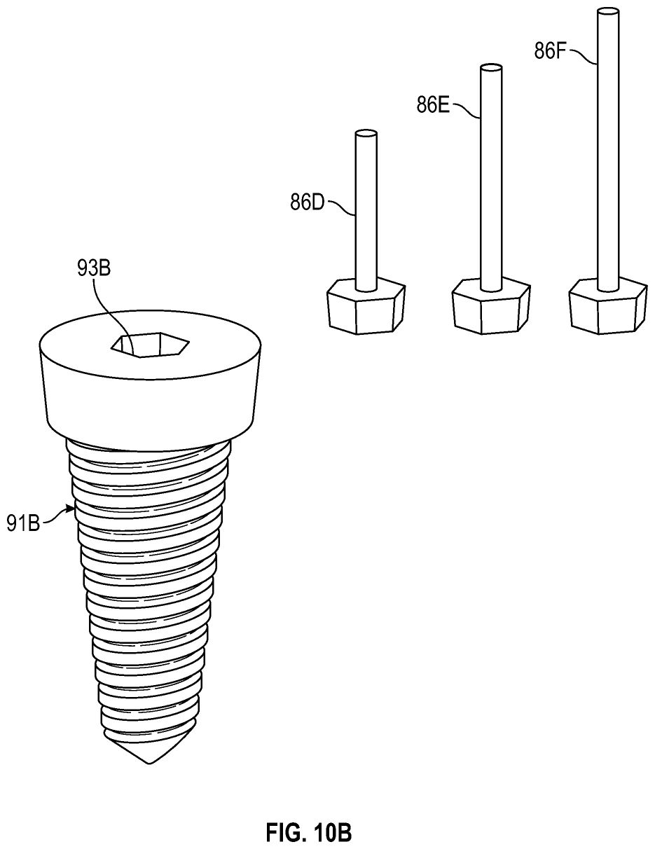

Referring to FIG. 6, a flow diagram 48 of an exemplary method is shown in accordance with the present disclosure. The method begins at block 50 with the step of determining a desired dental implant depth and/or diameter. This determination may be made by a dentist, surgeon or other operator based on a variety of factors including, and without limitation, observed patient anatomy, the diameter and/or length of available/chosen dental implants, and based on one or more radiological procedures of the patient (e.g. computed tomography scan, x-ray, etc.). Later, at block 52, a preparation drill bit 10 (FIGS. 1, 2 and 5) is selected based on the desired dental implant diameter and/or depth determined at block 50. The preparation drill bit 10 may be selected as having a desired length equal to the desired dental implant depth, or alternatively, with a length less than the desired dental implant depth; for example and without limitation, with a length smaller than the desired dental implant depth by 2 millimeters, 4 millimeters, 6 millimeters, 8 millimeters or 10 millimeters. As discussed above, the length of the selected preparation drill bit 10 may be equal to the sum of the length 13 of base portion 12 and length 16 of tip portion 14, or the length 16 of the tip portion 14 only. At block 53, the operator selects a shaft (discussed later herein, e.g. FIGS. 10A and 10B) based on anatomical constraints of the patient and/or based on the chosen preparation drill bit and chosen dental implant. Anatomical constraints may include, for example, the size and/or shape of the mouth of the patient. A larger size mouth may accommodate a larger length shaft to be selected by the operator, which may make the drilling with the shaft easier or more manageable by the operator. Conversely, a smaller size mouth may require or make desirable a smaller length shaft to be selected by the operator to make drilling with the shaft easier or more manageable. At block 54, the preparation drill bit 10 selected at block 52 is used to drill an initial bone cavity with, for example, a motor-equipped tool (or other actuator instrument). During drilling, the base portion 12 cuts the cortical bone to accommodate a diameter of an intended implant to be installed in the bone of the patient. In operation, the preparation drill bit 10 may pass through the cortical bone layer. The initial bone cavity is formed having a depth at the desired dental implant depth, or less than the desired dental implant depth, for example, 2 millimeters, 4 millimeters, 6 millimeters, 8 millimeters or 10 millimeters less than the desired dental implant depth.

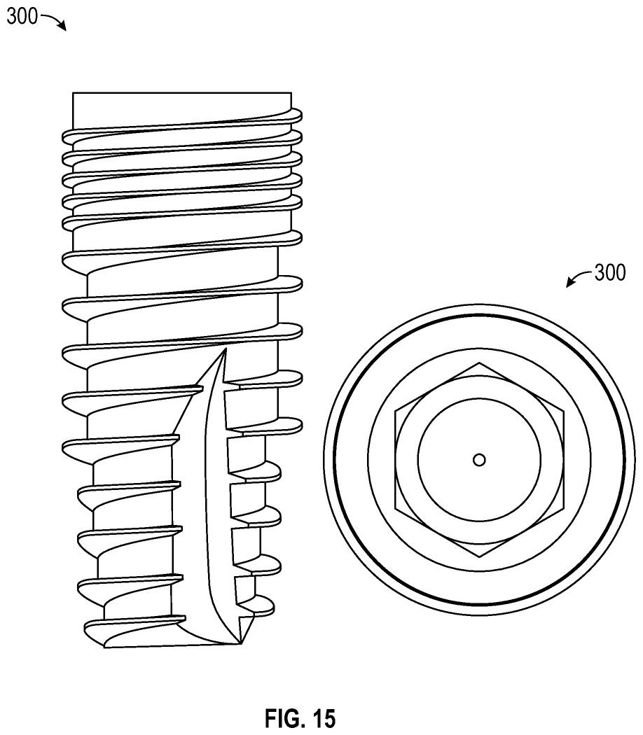

At block 56, a compression drill bit 26 is selected that corresponds to the preparation drill bit 10 selected at block 52 and may optionally be drilled using the same shaft used to drill with the preparation drill bit at block 52. The compression drill bit 26 may correspond to the preparation drill bit 10 by being the same or similar size (i.e. length in a longitudinal direction and/or a diameter across a face of the drill bit) At block 58, the compression drill bit 26 selected at block 56 is actuated to rotate (e.g. via the hand-piece used to actuate the preparation drill bit 10 to rotate) to substantially simultaneously cut or remove bone fragments from a bottom portion of the initial bone cavity at a medullary bone layer beneath the cortical bone layer and compress the bone fragments in a radial direction against a side wall of the initial bone cavity at a portion of the bone that contains the medullary bone layer thereby compressing the bone fragments and the bone side wall. At block 59, a dental implant (e.g. 300 in FIG. 15) is inserted or installed in the final bone cavity, and may optionally be inserted or installed using the same shaft used to drill with the preparation drill bit at block 54 and/or the same shaft used to drill with the compression drill bit at block 58. The dental implant may be inserted or installed with the motor-equipped tool (or actuator instrument) used in blocks 54 and 58, using a different actuator, or by hand with a hand tool that may optionally be configured to connect to the shaft used drill at blocks 54 and 58. While the step of selecting the compression drill bit 26 at block 56 has been shown as being after the step of drilling the initial bone cavity at block 54 with the preparation drill bit 10, it is within the scope of the present disclosure for the step of selecting the compression drill bit 26 as being before the drilling of the initial bone cavity with the preparation drill bit 10.

The substantially simultaneous drilling and compressing at block 58 transforms the initial bone cavity into a final bone cavity and is ready for dental implant insertion. The bone side wall of the final bone cavity has a bone density (or bone density gradient) that is greater than the bone density (or bone density gradient) of the initial bone cavity at an inner bone layer or medullary bone layer due to the compaction of the bone fragments at block 58. In some embodiments, there is only one preparation drill be used in the drilling of the preparation site for the dental implant. In some embodiments there may be optionally be pre-drilling of the preparation site before block 52 or block 54 with a preparation drill bit smaller than the preparation drill bit selected at block 52 in order to prepare the bone cavity before drilling using the preparation drill bit selected at block 52. Advantageously, the ability to use a maximum of one or two preparation drill bits to form an initial bone cavity in a patient before drilling with a compression drill bit is an improvement over conventional devices and methods that require many preparation drill bits, such as four to nine preparation drill bits, in order to form the initial bone cavity. However, the drill kits and methods of the present disclosure are not limited to applications of using only one or two preparation drill bits, more than two preparation drill bits may be used to drill the preparation site for the dental implant. There may be one or more cleaning and/or finishing steps to make the final bone cavity complete as a preparation site for a dental implant prior to insertion of the dental implant in the patient.

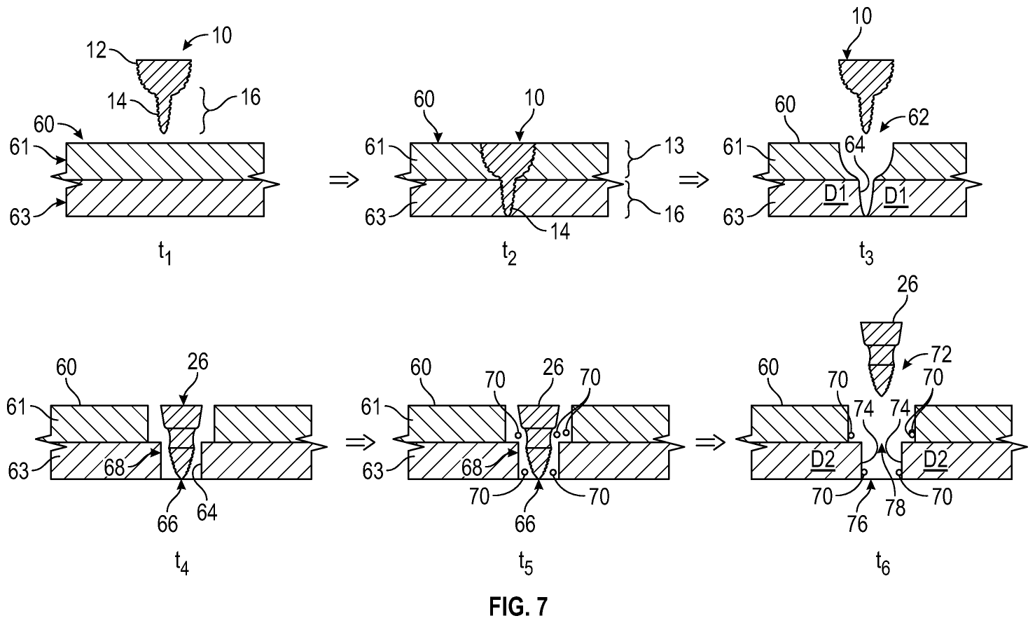

Referring to FIG. 7, an illustration is shown of a method in accordance with the present disclosure. At time t₁, a preparation drill bit 10 is shown above a bone 60 of a patient. The bone 60 of the patient having a cortical bone layer 61 and medullary bone layer 63. At time t₂, the preparation drill bit 10 is actuated or rotated by an actuator instrument (not shown) and the entire tip portion 14 is inserted into the bone 60. At time t₃, the preparation drill bit 10 is removed from the bone 60, leaving an initial bone cavity 62 formed in the bone 60 defined by a bone side wall 64. The initial bone cavity 62 has a bottom portion 66 and an upper portion 68. The density of the bone at the bone side wall 64 at the upper portion 68 has an initial bone density D₁ (or bone density gradient) at a medullary bone layer.

At time t₄, a compression drill bit 26 corresponding to the preparation drill bit 10 is inserted into the initial bone cavity 62. At time t₅, the compression drill bit 26 is actuated or rotated by an actuator instrument to substantially simultaneously cut or remove bone fragments and compress bone fragments as described herein. Bone fragments 70 are shown as simultaneously being cut or removed from the bottom portion 66 of the initial bone cavity 62 and forced in a radial direction to compress the bone fragments 70 against the bone side wall 64 at the upper portion 68. At time t₆, the bone fragments 70 are compressed against or into the bone side wall 64 and the compression drill bit 26 is removed from the bone 60, thereby forming a final bone cavity 72 formed by a bone side wall 74. The final bone cavity 72 has a bottom portion 76 and an upper portion 78. The density of the bone at the bone side wall 74 at the medullary bone layer has a final bone density D₂ (or bone density gradient). The final bone density D₂ (or bone density gradient) is greater than the initial bone density D₁ (or bone density gradient) at the medullary bone layer. The diameter of the base portion 12 of the preparation drill bit 10 may correspond to the diameter of an implant (e.g. dental implant) that will be inserted or installed in the final bone cavity 72, e.g. in the cortical bone layer 61.

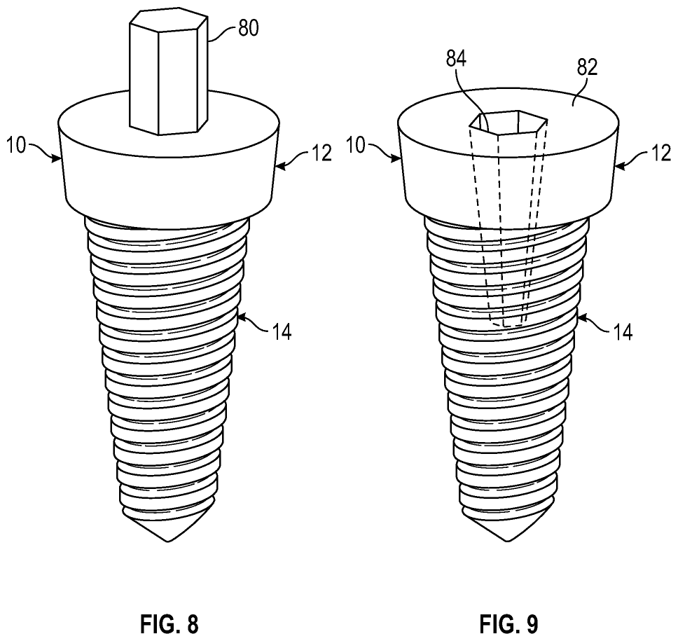

Referring to FIG. 8, a preparation drill bit 10 is shown with a connection geometry 80 in accordance with the present disclosure. The connection geometry 80 is a male connector protruding from a top side 82 of the base portion 12 of the preparation drill bit 10. The connection geometry 80 is configured to be received by a corresponding female connector of an actuator instrument or a shaft of an actuator instrument.

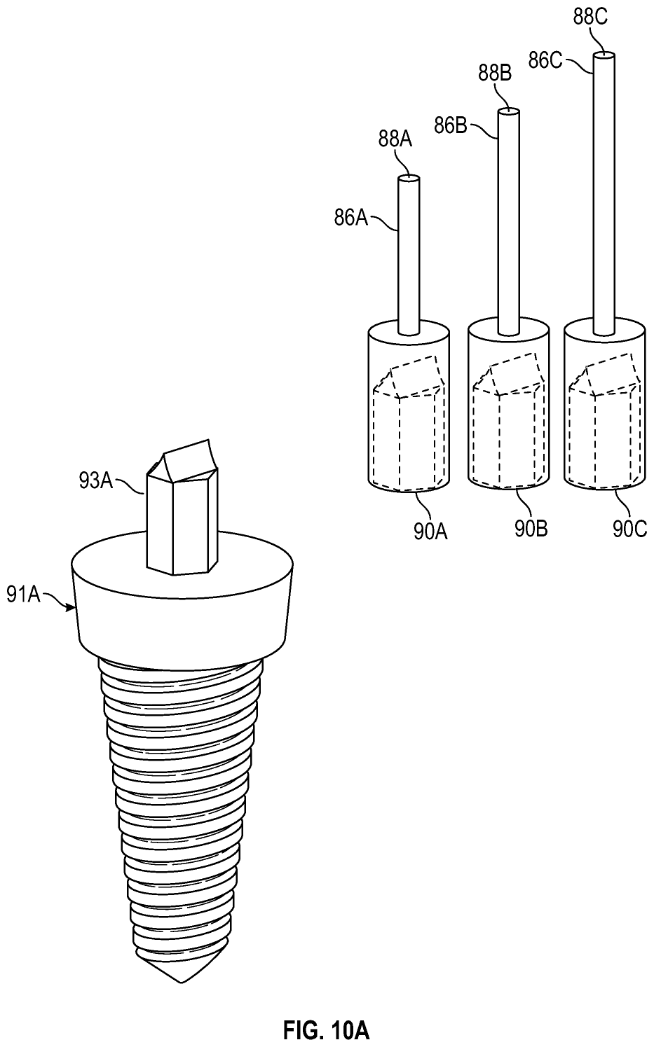

Referring to FIG. 9, a preparation drill bit 10 is shown with a connection geometry 84 in accordance with the present disclosure. The connection geometry 84 is a female connector defined within the base portion 12 of the preparation drill bit 10 and opening at a top side 82 thereof. The connection geometry 84 is configured to receive a corresponding male connector of an actuator instrument or a shaft of an actuator instrument.

While the connection geometries 80, 84 have been shown and described in connection with a preparation drill bit 10, it is within the scope of the present disclosure for the connection geometries to be applied with a compression drill bit 26. The connection geometries 80, 84 advantageously allow a plurality of preparation drill bits 10 and/or a plurality of compression drill bits 26 to be quickly interchanged with an actuator instrument so that a dentist or other use can quickly and efficiently change drill bits as desired, thereby reducing operating time. Further, the connection geometries may allow connection between a shaft connected to the actuator instrument and to the drill bit (10, 26) in a touch-less manner through different touch-less connection types, such as a friction fit (or interference fit) connection. This connection feature allows an operator to quickly and easily switch drill bits (10, 26) and/or shafts of the actuator instrument in a touch-less manner, which promotes sterility of the drill bits (10, 26) or system.

Referring to FIG. 10A, a plurality of shafts (or connectors) 86A, 86B, 86C (collectively or generally 86) are shown in accordance with the present disclosure. Each shaft 86 has a different length. The shafts 86 have a proximal end 88 configured for connection with an actuator instrument or hand-piece (e.g. a drill) and a distal end 90 configured for connection with connection geometries 80, 84 (shown in FIGS. 8 and 9) of a preparation drill bit 10 or a compression drill bit 26 as discussed herein. The plurality of shafts 86 may be included in a universal drill kit with the preparation drill bits 10 and compression drill bits 26 and provide the dentist or other user the option to have a drill bit at a desired length from the actuator instrument depending on the application being addressed. For example, a patient with a small mouth may require small shaft length. A universal drill kit in accordance with the present disclosure may have multiple preparation drill bits 10, compression drill bits 26 and shafts 86 with the same connection geometry type in order to interchangeably fit or attach to the same hand-piece for replacement during drilling. The shafts 86 are configured for connecting to a bit 91A with an external connection 93A (male connection geometry).

Referring to FIG. 10B, a plurality of shafts (or connectors) 86D, 86E, 86F similar to the shafts 86 of FIG. 10A are shown, but configured for connecting to a bit 91B with an inset connection 93B (female connection geometry). Accordingly, a universal drill kit may include a plurality of different shaft lengths to accommodate different types of drill bit connections (e.g. external connection and inset connection).

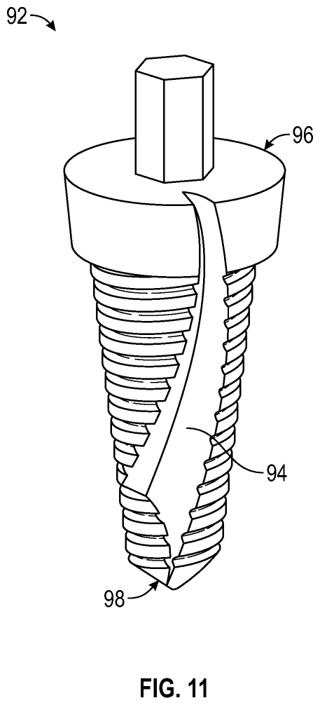

Referring to FIG. 11, a bone harvesting drill bit 92 is shown in accordance with the present disclosure. The bone harvesting drill bit 92 has an internal channel 94 extending from a proximal end 96 to a distal end 98 of the harvesting drill bit 92. The bone harvesting drill bit 92 is configured to extract bone fragments from a bone of a patient through the internal channel 94 from the distal end 98 to the proximal end 96 when the bone harvesting drill bit 92 is actuated or rotated by an actuator instrument. The bone harvesting drill bit 92 may advantageously be included in a universal drill kit or a universal drill system.

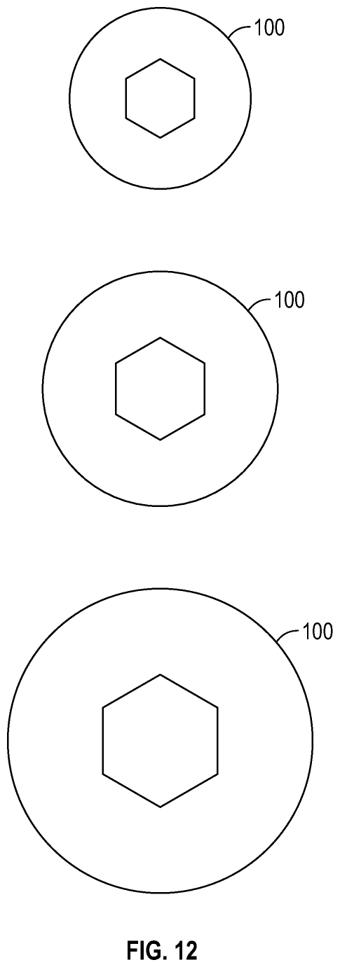

Referring to FIG. 12, a top view of a plurality of drill bits 100 with different diameters with shown in accordance with the present disclosure. The drill bits 100 may have different diameters and a short extension inset for different length shafts. The drill bits 100 are configured with a retentive design for shaft attachments for internal or external attachment.

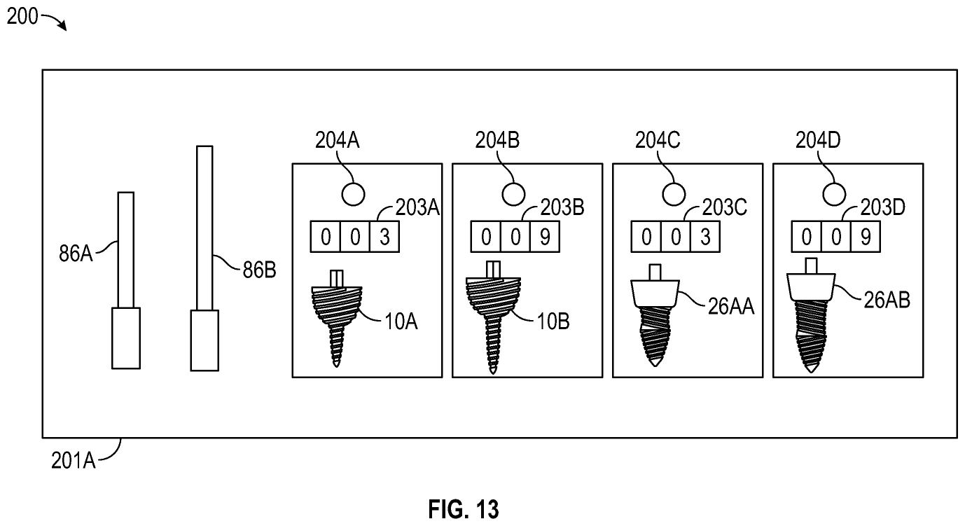

Referring to FIG. 13 a top view of a universal drill system or kit 200 is shown in accordance with the present disclosure. The drill kit 200 includes at least one shaft (or connector) 86A, 86B (FIG. 10A) of a first connection type, a first plurality of preparation drill bits 10A, 10B (FIG. 2), and a first plurality of compression drill bits 26AA, 26AB (FIG. 4) arranged in a container 201A. Each of the shafts 86 of the first connection type have a different length in a longitudinal extension direction of the shafts 86. Each of the shafts 86 of the first connection type has a female connection geometry configured to connect to a male connection geometry of each of the first plurality of preparation drill bits 10 and to a male connection geometry of the first plurality of compression drill bits 26. While FIG. 13 shows that there are two shafts 86, two drill bits 10 and two compression drill bits 26, it is within the scope of the present disclosure for the number of shafts 86, preparation drill bits 10 and compression drill bits 26 to be any number, such as one, three, five, ten, etc. Further, the number of shafts 86, preparation drill bits 10 and compression drill bits does not need to correspond in number. For example, a drill kit according to the present disclosure may comprise one shaft 86, four preparation drill bits 10 and five compression drill bits 26.

Each drill bit 10, 26 may arranged to be secured in the container 201A near, or in association with, a respective counter 203A, 203B, 203C, 203D (collectively or generally 203). The counters 203 display a number(s). The number(s) displayed by the counters 203 is configured to be manipulated or operated by a respective input element 204A, 204B, 204C, 204D (collectively or generally 204). A user of the drill kit 200 can manipulate and/or operate the input element(s) 204 to reflect an amount of usage of the drill bit 10, 26 near or associated with the counter 203. The counter 203 thereby may reflect a usage amount of each drill bit 10, 26, analogous to a vehicle mileage odometer. The counter 203 provides a means for the operator to know when a drill bit should be sharpened, replaced or refurbished. The counter 203 may be configured to display a number of times used (number of discrete instances utilized, such as with different patients), or a duration of use (e.g. hours). Each drill bit 10, 26 in the drill kit 200 may have a counter 203, or only some of the drill bits 10, 26 may have a counter 203. For example, only preparation drill bits 10 have a respective counter 203, or only compression drill bits 26 have a respective counter 203. In this embodiment, the input element 204 are buttons, but any form of input element may be utilized, for example and without limitation, knobs, sliders, electronics, dials, or the like. In some embodiments, the input element 204 is integral with the counter 203, for example, the numbers of the counter are adjustable by an operator and constitute the input element, or the numbers are a touch screen electronic device configured to be adjusted by the operator.

While the compression drill bits 26 are shown with the concave compression geometry (34 of 26A in FIG. 3), it is within the scope of the present disclosure for the compression drill bits to have any known compression geometry, such as, for example, the straight compression geometry (34 of 26B in FIG. 3) with thread grooves. Other types of preparation drill bits and/or compression drill bits are within the scope of the present disclosure.

While the connection geometries of the shafts 86 have been shown and described as being female and the connection geometries of the drill bits 10, 26 have been shown and described as being male, it is within the scope of the present disclosure for drill kits 200 to have the opposite connection geometry. In other words, the shafts 86 may have a male connection geometry that are configured to connect to female connection geometries of the drill bits 10, 26 (and/or dental implant as discussed herein).

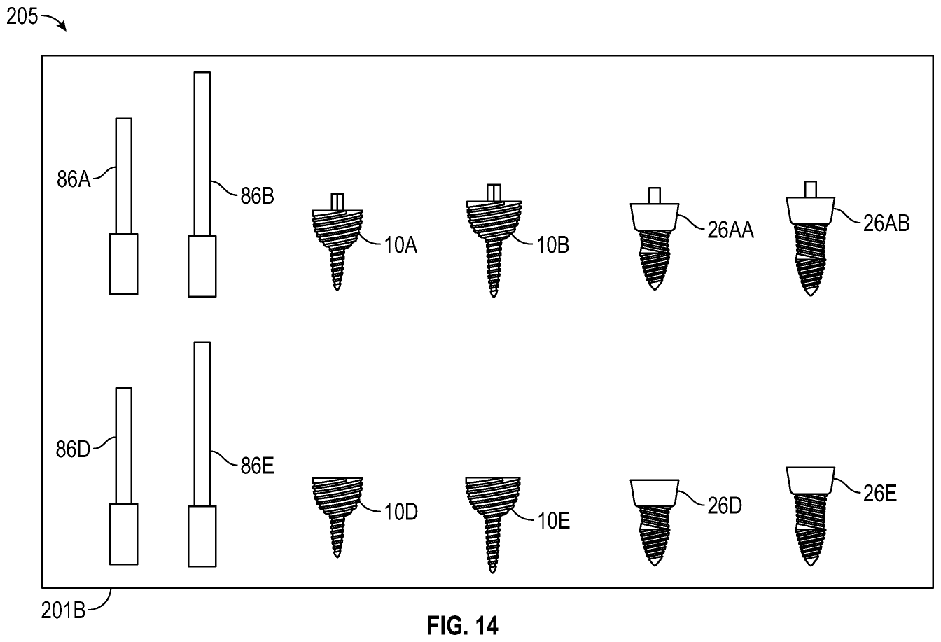

Referring to FIG. 14, a top view of a universal drill system or kit 205 is shown in accordance with the present disclosure. The drill kit 205 is similar to the drill kit 200 of FIG. 13 in that the drill kit 205 includes at least one shaft (or connector) 86A, 86B (FIG. 10A) of a second connection type, a first plurality of preparation drill bits 10A, 10B (FIG. 2), and a first plurality of compression drill bits 26AA, 26AB (FIG. 4) arranged in a container 201B. The drill kit 205 further includes a second plurality of shafts 86D, 86E, a second plurality of preparation drill bits 10A, 10B, and a second plurality of compression drill bits 26AA, 26AB arranged in the container 201B. Each of the shafts 86D, 86E of the second connection type have a different length in the longitudinal extension direction of the shafts. Each of the shafts 86D, 86E of the second connection type has a male connection geometry configured to connect to a female connection geometry of each of the second plurality of preparation drill bits 10D, 10E and to a female connection geometry of each of the second plurality of compression drill bits 26D, 26E.

In this embodiment, the shafts 86A, 86B of the first connection type having a female connection geometry cannot operatively connect to the female connection geometry of the second plurality of preparation drill bits 10D, 10E or to the female connection geometry of the second plurality of compression drill bits 26D, 26E. Similarly, the shafts 86D, 86E of the second connection type having a male connection geometry cannot operatively connect to the male connection geometry of the first plurality of preparation drill bits 10A, 10B or to the male connection geometry of the first plurality of compression drill bits 26AA, 26AB. In some embodiments, the connection geometries of both connection type shafts may be similar in that both connection geometries are both male or are both female, but the connection geometries are different such that the shafts of the first connection type cannot operatively connect to the second pluralities of preparation drill bits and compression drill bits, and the shafts of the second connection type cannot operatively connect to the first pluralities of preparation drill bits and compression drill bits. Accordingly, it will be recognized by those of ordinary skill in the art that connection geometries of different connection types of shafts may be both male or both female, but still different such that the different shafts are configured to operatively connect to different sets of preparation drill bits and/or compression drill bits (and/or dental implants as is discussed below).

Referring to FIG. 15, a diagram of a dental implant 300 is shown in accordance with the present disclosure. The dental implant 300 has a connection geometry at a top face that is configured to correspond with one or more of the shafts 86 of a drill kit (e.g. 200 of FIG. 13 or 205 of FIG. 14). The shaft 86 is configured to operatively connect to the dental implant 300 at the connection geometry, e.g. via a friction fit (or interference fit). The dental implant 300 may be inserted or installed in a bone site through actuation of the shaft 86 via a drill (or other actuator) or a hand tool configured to connect to the shaft 86 (or dental implant 300) and rotated or actuated by hand by an operator. One or more dental implants 300 may be part of a drill kit (e.g. 200 FIG. 13 or 205 FIG. 14) and be arranged in the container of the drill kit, or packaged separately from the container containing the shaft(s), preparation drill bit(s) and/or compression drill bit(s).

Advantageously, one or more shafts of a drill kit or method according to the present disclosure may be configured to operatively engage with each of a preparation drill bit, a compression drill bit and a dental implant. The ability to have the same shaft connect to all three of these elements allows the operator to efficiently perform a dental procedure, such as an osteotomy. Optionally, the shaft connection and disconnection from each of these elements may be performed in touch-less manner, for example, if the connection types are friction fit (or interference fit) type. Thus, an operator can connect a shaft to a preparation drill bit for drilling (e.g. through a friction fit), then disconnect the shaft from the preparation drill bit in a touch-less manner before connecting the same shaft to a compression drill bit (e.g. through a friction fit). Similarly, after finished drilling with the compression drill bit, the operator may disconnect the shaft from the compression drill bit in a touch less manner before optionally connecting the shaft to a dental implant for insertion into the patient (e.g. through a friction fit). Touch-less manner means the operator does not need to touch either of the preparation drill bit, compression drill bit and/or dental implant with his or her hands, which promotes sterility. The ability to connect with a friction fit allows for a solid connection of all parts to reduce wobbling and easy acceptance when connected, and easy removal/disconnection when changing between bits during site preparation.

In some embodiments, a preparation drill bit 10 has a length 16 of tip portion 14 of 8 mm, 10 mm, 11.5 mm, 13 mm or 16 mm. In some embodiments, the preparation drill bit 10 has a width 18 of 3.0 mm, 3.5 mm, 4.3 mm, 5.0 mm or 6.0 mm. In some embodiments, a compression drill bit 26 has a cutting tip 28 and compression section 32 with a length sum 35 of 8 mm, 10 mm, 11.5 mm, 13 mm or 16 mm. In some embodiments, the compression drill bit 26 has a maximum width 37 of 3.0 mm, 3.5 mm, 4.3 mm, 5.0 mm or 6.0 mm.

While the preparation drill bits 10, compression drill bits 26 and dental implant 300 have been shown and described with a particular shape or groove/flute patterns (e.g. at the cutting tips or compression sections of the bits or for the gripping surfaces of the implants), it is within the scope of the present disclosure for the preparation drill bits 10, compression drill bits 26 and dental implants 300 to have any shape or groove/flute pattern as is known in the art to perform the functions thereof. Additionally, the direction of the groove/flute patterns may be reversed and perform substantially the same way if actuated or rotated in the reverse direction, for example, by a drill (or other actuator). Thus, the particular shapes and groove/flute patterns shown and described in the present disclosure are not limiting on the claims of the present application unless the claims are recite a particular shape or groove/flute pattern.

Advantageously, apparatuses, methods and systems of the present disclosure can laterally increase bone density at a medullary bone layer while simultaneously drilling the final bone cavity for the dental implant. The increased bone density in the medullary bone layer due to lateral bone compression provides increased initial stability for a dental implant. Such apparatuses, methods and systems are especially advantageous for use with patients having below normal bone density at a site for dental implant.

Advantageously, apparatuses, methods and systems of the present disclosure can drill to a desired depth and diameter quickly and more efficiently than conventional drilling methods while simultaneously providing greater initial stability for a dental implant.

Advantageously, a universal drill system having a plurality of shafts with different lengths may be easily selectable and easy to pick up from a drill kit containing the universal drill system.

Advantageously, systems, kits and methods according to the present disclosure provide for a more efficient way to remove cortical bone of a patient to a chosen diameter and densifying the trabecular bone in fewer steps and with greater sterility than achieved with conventional devices and methods. In some embodiments, the actuator may actuate or rotate the shaft and drill bit with a low-speed, high-torque motion, which may be advantageous in situations where only one or two preparation drill bits are used to drill the initial bone cavity because excess heat may be avoided during the drilling that may be detrimental to the success or operation of the dental implant. In contrast to conventional drill bits that have integral shafts, the drill bits of the present disclosure may not have integral shafts and the shafts of the present disclosure are interchangeable to allow connection to a variety of different types and sizes of drill bits, e.g. different sized preparation drill bits and to different sized compression drill bits. However, the novel methods according to the present disclosure may also be performed with conventional drill bits that do have integral shafts. Specifically, a conventional preparation drill bit may be utilized to drill the initial bone cavity at the desired size/diameter for the intended dental implant through low-speed, high-torque rotation of the conventional drill bit (or two conventional preparation drill bits if needed/desired depending on the intended size of the initial bone cavity). As discussed above, the methods of the present disclosure are advantageous over conventional methods that utilize many preparation drill bits to form the initial bone cavity (e.g. four to nine preparation drill bits). Moreover, since the drill bits of the present disclosure may not have integral shafts, different length shafts may be connected to the same drill bit allowing an operator to select the desired length shaft for that drill bit, which is an improvement over conventional drill bits in which a shorter length shaft is not an option for a drill bit having an integral shaft that is longer than desired.

The apparatuses and methods according to the present disclosure advantageously allow for procedures to be performed (e.g. dental procedures) with less steps, for example, less drilling steps. As discussed above, instead of performing many drillings with a preparation drill bit to form a cavity (e.g. bone cavity) at the desired diameter, an operator can choose the preparation drill bit at the desire diameter and perform a preparation drill step only once. The determination of the appropriate sized preparation drill bit that corresponds to the chosen implant (e.g. dental implant) may be chosen based on information available to the operator, for example, through cone beam, computed tomography scan (i.e. “CT scan”), x-ray detection, or other radiological procedures or equipment.

While the present disclosure has been illustrated with respect to particular embodiments thereof, it should be appreciated by those of ordinary skill in the art that various modifications to this disclosure may be made without departing from the spirit and scope of the present disclosure.

What is claimed is:

1. A drill kit for installing a dental implant comprising:
at least one shaft of a first connection type, each shaft of the at least one shaft of the first connection type having a different length than each other shaft of the at least one shaft of the first connection type;
a first plurality of preparation drill bits each having a first connection geometry and each having a different size; and
a first plurality of compression drill bits each having the first connection geometry and each having a different size;
wherein each shaft of the at least one shaft of the first connection type has a proximal end configured to connect with an actuator instrument and a distal end configured to connect to the first connection geometry of each of the first plurality of preparation drill bits and to the first connection geometry of each of the first plurality of compression drill bits.

2. The drill kit according to claim 1, wherein the different size of each of the first plurality of preparation drill bits is, at least in part, a different diameter.

3. The drill kit according to claim 1, wherein the at least one shaft comprises two shafts.

4. The drill kit according to claim 1, further comprising a dental implant having the first connection geometry, wherein each shaft of the at least one shaft of the first connection type is configured to connect to the first connection geometry of the dental implant.

5. The drill kit according to claim 1, wherein each of the first plurality of preparation drill bits are configured to drill bone when actuated by an actuator via a shaft of the at least one shaft of the first connection type.

6. The drill kit according to claim 1, wherein each of the first plurality of compression drill bits are configured to compress bone fragments into a side wall of a bone cavity when actuated by an actuator via a shaft of the at least one shaft of the first connection type.

7. The drill kit according to claim 1, further comprising:
at least one shaft of a second connection type, each shaft of the at least one shaft of the second connection type having a different length from each other;
a second plurality of preparation drill bits each having a second connection geometry and each having a different size; and
a second plurality of compression drill bits each having the second connection geometry and each having a different size;
wherein each shaft of the at least one shaft of the second connection type has a proximal end configured to connect with the actuator instrument and a distal end configured to connect to the second connection geometry of each of the second plurality of preparation drill bits and to the second connection geometry of each of the second plurality of compression drill bits; and
wherein the second connection geometry is different than the first connection geometry.

8. The drill kit according to claim 7, wherein the first connection geometry is a male connection geometry.

9. The drill kit according to claim 8, wherein the second connection geometry is a female connection geometry.

10. The drill kit according to claim 7, wherein each shaft of the at least one shaft of the first connection type cannot operatively connect to the second connection geometry, and wherein the at least one shaft of the second connection type cannot operatively connect to the first connection geometry.

11. The drill kit according to claim 1, further comprising a counter configured to display a number, where the number displayed by the counter is adjustable by an operator of the drill kit.

12. The drill kit according to claim 1, further comprising a plurality of counters configured to display a number, wherein each counter of the plurality of counters is associated with a respective drill bit of the first plurality of preparation drill bits and/or the first plurality of compression drill bits, and wherein the number displayed by each counter are adjustable by an operator of the drill kit.

13. The drill kit according to claim 1, wherein the distal end of each shaft of the at least one shaft of the first connection type is configured to connect to the first connection geometry of each of the first plurality of preparation drill bits and to the first connection geometry of each of the first plurality of compression drill bits through a friction fit.

14. The drill kit according to claim 1, wherein each compression drill bit of the plurality of compression drill bits comprises:
a base portion;
a compression section; and
a cutting tip;
wherein the cutting tip is configured to cut or remove bone fragments from a bone of a patient when the compression drill bit is rotated; and
wherein the compression section is configured to force at least some of the bone fragments cut or removed by the cutting tip in a radial direction, thereby compressing the bone fragments in a side wall of the bone.

15. A method of drilling a preparation site for a dental implant comprising:
determining a desired dental implant diameter and/or depth;
selecting a preparation drill bit based on the determined desired dental implant diameter and/or depth;
drilling a patient bone of a patient using the selected preparation drill bit, thereby forming an initial bone cavity;
selecting a compression drill bit corresponding to a size of the selected preparation drill bit;
drilling the patient bone using the selected compression drill bit;
wherein no more than two preparation drill bits are used to form the initial bone cavity.

16. The method according to claim 15, wherein the drilling the patient bone using the selected preparation drill bit is performed using a shaft; and wherein the drilling the patient bone using the selected compression drill bit is performed using the shaft.

17. The method according to claim 15, wherein no more than one preparation drill bit is used to form the initial bone cavity.

18. The method according to claim 15, further comprising:
selecting a shaft based on anatomical constraints of the patient before the drilling the patient bone using the selected preparation drill bit;
connecting the preparation drill bit to the shaft before the drilling the patient bone using the selected preparation drill bit; and
connecting the compression drill bit to the shaft before the drilling the patient bone using the selected compression drill bit.

19. The method according to claim 15, wherein the drilling the patient bone using the selected compression drill bit comprises:
cutting or removing bone fragments from a bottom portion of a bone cavity of a bone, the bone cavity being defined by a bone side wall of the patient bone; and
forcing at least some of the bone fragments in a radial direction at an upper portion of the bone cavity, thereby compressing the at least some of the bone fragments into the bone side wall.

20. A method of installing a dental implant comprising:
determining a desired dental implant diameter and/or depth;
selecting a preparation drill bit based on the determined desired dental implant diameter and/or depth;
drilling a patient bone of a patient using the selected preparation drill bit, thereby forming an initial bone cavity;
selecting a compression drill bit corresponding to a size of the selected preparation drill bit;
drilling the patient bone using the selected compression drill bit, thereby forming a final bone cavity;
installing a dental implant in the final bone cavity;
wherein the drilling the patient bone using the selected preparation drill bit is performed using a shaft;
wherein the drilling the patient bone using the selected compression drill bit is performed using the shaft; and
wherein the installing the dental implant is performed using the shaft.
